(19) United States
(12) Patent Application Publication
(10) Pub. No.: US 2019/0015191 A1
(43) Pub. Date: Jan. 17, 2019

(54) PROSTHETIC AORTIC ROOT REPLACEMENT GRAFT

(71) Applicant: Denis BERDAJS, Oberwil (CH)

(72) Inventor: Denis BERDAJS, Oberwil (CH)

(21) Appl. No.: 15/651,482

(22) Filed: Jul. 17, 2017

(51) Int. Cl. A61F 2/06; A61L 27/50; A61F 2/24

(52) U.S. Cl. CPC A61F 2/06; A61L 27/40; A61F 2/2412; A61L 27/507

(57) ABSTRACT

A prosthetic aortic root replacement graft for preserving the native aortic valve that includes a biocompatible flexible material having
a) a tubular superior segment (3), with an upper free edge (2) for anastomosis of the prosthesis to the aortic arch, and a lower edge (4);
b) a hollow medial segment (5) with an upper edge (6) that is connected to the lower edge (4) of the superior segment (3) and a lower edge (8); and
c) optionally a tubular inferior segment (10).

The medial segment (5) includes three triangular-like tongues (7) tapering towards the upper edge (6) and three bulges (12). The bulges (12) and the tongues (7) are in an alternating arrangement.

### BACKGROUND OF INVENTION

### 1. Field of Invention

The invention relates to a prosthetic aortic root replacement graft for preserving the native aortic valve. It is intended for being implanted during a valve sparing procedure, in order to replace the aortic root and ascending aorta.

The field of invention is cardiovascular surgery, more specifically where the diseased aortic root (type A dissection, Marfan and or Loeys-Dietz syndrome) and the ascending aorta are replaced. The architecture of the proposed new graft is based on morphological and physiological principles in the native aortic root with normal shaped sinuses of Valsava, contributing to normal opening and closure of the three leaflets.

The actual surgical strategy in most cases for aortic root dilative pathology is implantation of a mechanical or biological composite graft. In younger patients here the long-term durability of the mechanical graft/prosthesis is used with the price of long life anticoagulation and to its related complications. However, from the early nineties, two reconstructive procedures are taking place in routine daily practice (David resp. reimplantation technique and Yacoub resp. remodelling technique) in order to address younger patients with systematic diseases, such as Marfan and Loeys-Dietz, where the main intention is to avoid the disadvantages of the mechanical or biological graft/prosthesis. Although the remodelling procedure seems to mimic the natural aortic root geometry and physiology in a more accurate manner, the clinical data on valve function and re-intervention rate are barley comparable with the re-implantation technique. Long-term results for the David procedure with freedom from valve/cusp related re-intervention is around 80% (David T E. Aortic valve sparing operations: outcomes at 20 years. Ann Cardiothorac Surg. 2013; 2:24-9), whereas for the Yacoub procedure the reoperation rate in the mid-term is 17% (de Oliveira N C et all. Results of surgery for aortic root aneurysm in patients with Marfan syndrome. J Thorac Cardiovasc Surg. 2003; 125: 789-796). Failures in both reconstructive procedures are mainly caused by important valve dysfunction. The excellent clinical outcome of the re-implantation technique provides hemodynamically less favorable conditions as compared to the remodeling procedure (Wentzel J J et al. Endothelial shear stress in the evolution of coronary atherosclerotic plaque and vascular remodeling: current understanding and remaining questions. Cardiovasc Res. 2012: 96:234-43).

In order to be able to use the new surgical procedure a new prosthetic aortic root replacement graft is disclosed that provides the clinical advantages of re-implantation (stabilization of the aortic root base) and includes the hemodynamical superiority of the remodeling procedure. Additionally, the disclosed new device is easy to be used, promotes wider acceptance in the surgical community so that it may not be only used in elective cases, but also in salvage interventions such as for example in type A aortic dissection.

### 2. Brief Description of Related Art

Various aortic root prosthesis for being implanted into a patient during a valve spearing surgery as a replacement for a biological aortic root segment of an ascending aorta are known in prior art, e.g. from Thubrikar, et al., U.S. Pat. No. 6,544,285 B1.

In the re-implantation technique (David procedure), where usually a straight graft is used, the intervalvular triangles providing the aortic leaflets attachment, are re-implanted in vertical direction. This does provide a natural position of the leaflets. In this way sufficient coaptation (closure surface) between the three leaflets is provided and with this also the one of the most important predisposition for long term functionality is fulfilled. However, the straight tube of such straight grafts does not have sinus part. The sinuses of Valsava are crucial for the normal function of the aortic leaflets. It was proven that the absence of the sinuses augment the pressure as well the shear stress on the leaflets surface, condition that in long term leads to the valve degeneration and failure of the aortic valve closure. Using sinus Valsava graft (De Paulis, Pub. No. US 2001/0049553 A1) where the aortic root is not re-implanted into a straight tube but is re-implanted in a bulging segment, the natural role of the Sinuses of Valsalva as such is provided. The Sinus Valsava grafts have a concave wall. Re-implanting the natural intervalvular triangles in this concave manner does not mimicking the natural vertical position of the leaflets attachment. In Valsalva grafts namely, the intervalvular triangles and with this the leaflets attachment are sewn in a spherical configuration to the wall of the sinus Valsava prosthesis (Hargreaves, Pub. No. US 2013/0226286 A1). This non-vertical positioning of the leaflets attachments results in augmentation of intercommissural distances and consequent leaflet restriction, respectively significant reduction in leaflet coaptation (or closure surface), a situation that even in mid term leads to aortic valve incompetence. One can also understand why the sinus Valsalva was not broadly accepted in aortic root repair procedures.

Aortic root remodeling procedure with restoration of the sinuses is, in some instances, intuitively more physiological when compared with the re-implantation technique (Leyh R G, et al. Opening and closing characteristics of the aortic valve after different types of valve-preserving surgery. Circulation 1999; 100:2152-60.). This is probably due to the preservation of the aortic root base physiology; however, the late dilatation of the aortic root base and consequent need for re-intervention is a major drawback of this method. This procedure was first described in the last century by Yacoub et al. (Yacoub M H, et al., Results of valve conserving operations for aortic regurgitation. Circulation 1983; 68:3-32) and is nowadays in general an acceptable method for repair of the aortic root dilatation in elderly patients with normal aortic root base dimension (David T E, Aortic root aneurysm: principles of repair and long term follow-up. J Thorac Cardiovasc Surgery 2010; 140 (6 Suppl): S14-9;). The graft prosthesis used for the remodeling procedure, per se does not include, the elements for stabilization of the aortic root annulus (Thubrikar, et al., U.S. Pat. No. 6,544,285 B1), and provides just a replacement of the ascending aorta and sinuses of Valsava. However using the mentioned prosthesis the intervalvular triangles as well the aortic root base rest native, without any support of the implanted graft. This situation especially in younger adults with degenerative disease such as Marfan syndrome, the aortic root base as well the intervalvular triangles in long term undergo structural changes and dilatation. Later this would result in restriction of the leaflet mobility, lack of the competent leaflets coaptation (or closure surface) and with this insufficient function which results in re-intervention rate of almost 20% in mid-term following the mentioned procedure. The lack of stabilization of the aortic root base and of the intervalvular triangles basically leads to failure of the Yacoub procedure.

According to De Paulis, Pub. No. US 2001/0049553 A1, and Hargreaves, Pub. No. US 2013/0226286 A1, the intervalvular triangles of the native aortic root are re-implanted in the spherical wall of the prosthesis. This is in contrast to the natural vertical direction/position of the native intervalvular triangles. The spherical/concave re-implantation of the intervalvular triangles in the Sinus Valsalva prosthesis (De Paulis, Pub. No. US 2001/0049553 A1, and Hargreaves, Pub. No. US 2013/0226286 A1) brings the intervalvular triangles in spherical position. However, the natural position of the intervalvular triangles is vertical, in order to assure optimal intercomissural distance as well correct leaflet coaptation and valve closure at diastole. The spherical position resp. non-vertical positioning of the intervalvular triangles and with this also of the leaflets attachment results in augmentation of the intercomissural distances and the reduction of the leaflet coaptation. This leads to the valve incompetence, and need for re-intervention.

According to Thubrikar, et al., U.S. Pat. No. 6,544,285 B1 the intervalvular triangles as well the aortic root base rest native, without any support of the implanted graft. This in younger adults with degenerative disease such as Marfan and Loyes-Dietz syndrome, the aortic root base as well the intervalvular triangles in long term undergo structural changes and dilatation.

This results in dilatation of the mentioned elements, and restriction of the leaflet mobility, lack of the competent leaflets closure during diastole and clearly in insufficient function of the leaflets which results in re-intervention rate of almost 20% in mid term following the mentioned procedure.

What is therefore needed is an improved prosthetic aortic root replacement graft that provides:
1) stabilization of the elements prone for dilatation such as of the intervalvular triangles, and of the aortic root base;
2) positioning to the intervalvular triangles in their almost natural like vertical position and with this restoration of the aortic leaflets geometry with sufficient coaptation;
3) circular stabilization of the aortic root base;
4) spherical components in its geometry, in order to replace the position and function of sinus of Valsava. This component that provides the convexity at the position of the sinus has horizontal expansion possibility and provides with outside bulging's the almost native like function of the aortic root sinuses; and
5) Regaining the normal physiological function (hemodynamics) after the surgical intervention of the re-implanted aortic valve.

### BRIEF SUMMARY OF THE INVENTION

It is an object of the invention to provide a prosthetic aortic root replacement graft which allows for more physiological solution as compared to prior art (e.g. Thubrikar, et al., U.S. Pat. No. 6,544,285 B1) by using the advantages of both hemodynamically superior remodeling and the excellent durability of the re-implantation techniques.

The invention solves the posed problem with a prosthetic aortic root replacement graft comprising a biocompatible flexible material, comprising
a) a tubular superior segment (3), with an upper free edge (2) for anastomosis of the prosthesis to the aortic arch, and a lower edge (4);
b) a hollow medial segment (5) with an upper edge (6) that is connected to the lower edge (4) of the superior segment (3) and a lower edge (8); and
c) optionally a tubular inferior segment (10);
   wherein the medial segment (5) comprises three triangular-like tongues (7) tapering towards the upper edge (6) and three bulges (12), whereby the bulges (12) and the tongues (7) are in an alternating arrangement.

Integrating convexity with role of sinuses of valsava is providing more physiological leaflet function, with implementation of the vertical triangular quasi rigid posts serving as spots for the re-implantation of the natural intervalvular triangles, resulting in almost natural vertical oriented position of the leaflet attachment as well of the three commissures. And with this the natural, optimal conditions for leaflets coaptation is provided.

Further suturing the intervalvular triangles at the triangular post of the graft/prosthesis also prohibits the dilatation of the natural intervalvular triangles, especially in degenerative disease.

In comparison with straight tube grafts, the graft according to the invention brings the replacement of the natural sinus of Valsava with convexity between the three triangular posts. Following the re-implantation these bulgings provide smooth and natural like valve function. In this way, a more natural, safe, durable and easily reproducible solution for young patients with aortic root disease is achieved.

The graft according to the invention includes a superior tubular part, where the distal part is for being attached to the aortic arch. The middle segment that is in direct continuation to the tubular part serves with its three triangular parts for natural fixation of the three intervalvular triangles and the three sinuses, each triangle of the middle segment is placed in vertical direction, includes an inner support and provides the natural vertical position of the aortic valve attachment after the valve re-implantation. The three sinuses are of convex contour, and thereby create a space between the open valve and the corresponding sinus in order to prevent the impact between the inner sinus wall and the leaflet as well provide with its ability to deform in horizontal direction more physiological almost natural hemodynamics of the re-implanted aortic root.

The optional inferior tubular segment is intended for attachment to the aortic root base.

The advantage(s) of the graft according to the invention are the following:
The graft definitively provides a more physiological solution as compared to the mentioned prior art devices;
   Integrating convexity with role of sinuses of Valsava is providing more physiological leaflet function,
   A natural vertical oriented position of the leaflet attachment as well of the three commissures, and
   Further suturing the intervalvular triangles at the triangular post of the graft also prohibits the dilatation of the natural intervalvular triangles, especially in degenerative disease.
In comparison with straight tube grafts, the graft according to the invention brings the replacement of the natural sinus of Valsalva with convexity between the three-triangular posts. Following the re-implantation these bulging's provide smooth and natural like valve function. In this way, a more natural, safe, durable and easily reproducible solution for young patients with aortic root disease is achieved.

Further advantageous embodiments of the invention can be commented as follows:

The aortic root prosthesis may comprise a tubular inferior segment 10, with a free lower edge 9 for fixation at the aortic root base upper surface, and an upper edge 11 that is connected to the lower edge 8 of the medial segment 5. The tubular inferior segment is positioned at the level of the aortic root base and provides aortic root annulus stabilization and prohibits the annulus dilatation with time, which is a well known phenomenon in Marfan and Loyes-Dietz syndrome.

The middle axis of the superior, medial and inferior segments 3, 5 and 10 may define a longitudinal axis 1 of the prosthesis.

In a special embodiment the three triangular-like tongues 7 extend from that the lower edge 8 of the medial segment 5 towards the upper edge 6 of the medial segment 5 in vertical direction.

The three triangular-like tongues 7 may essentially running parallel to the longitudinal axis 1. The three triangular-like tongues 7 are preferably reaching the upper edge 6 of the medial segment 5. The triangular-like tongues 7 may preferably be reinforced by wire-like elements 15 being positioned in the interior of the tongues 7. The wire-like elements 15 may have a semilunar shape.

In a special embodiment the three triangular-like tongues 7 are stiffer in structure compared to the three bulges 12. The three triangular-like tongues 7 are purposefully suitable for fixation to the intervalvular triangles of the native aortic root.

In a further embodiment the superior and inferior segments 3, 10 are elastically expandable in direction of the longitudinal axis 1.

In a further embodiment the three bulges 12 of the medial segment 5 are elastically expandable transversally to the longitudinal axis 1.

In a further embodiment the biocompatible flexible material has a textile structure, which preferably is warp-knitted or woven. The textile structure may be based on a polyester. The textile structure may be impregnated with an absorbable protein.

In a further embodiment the biocompatible flexible material comprises a GORETEX material.

In a further embodiment the graft according to the invention exhibits three symmetric planes having the middle axis 1 in common.

In a special embodiment the radius R₀ of the straight cylinder representing the superior segment 3 is larger than 13 mm. The radius R₀ of the straight cylinder representing the superior segment 3 is preferably smaller than 17 mm.

In a special embodiment the radius R₁ of the tubular sinus part of the graft in the middle of the middle segment 5 is in the range of 1.1 R₀ to 1.3 R₀ and typically is 1.2 R₀.

The height h₁ of the inferior segment 10 is preferably in the range of 3.6 R₁ to 4.5 R₁ and typically is 4×R₁. The height h₁ of the inferior segment 10 is purposefully larger than 5 mm, preferably larger than 8 mm. The height h₁ of the inferior segment 10 is purposefully smaller than 15 mm, preferably smaller than 12 mm.

In a further embodiment the height h₂ of the medial segment 5 is smaller than 15 mm and preferably smaller than 14 mm. The height h₂ of the medial segment 5 is purposefully larger than 10 mm and preferably larger than 11 mm.

In a further embodiment the length of the superior segment 3 is between 20 and 35 cm. The length of inferior segment 10 is purposefully between 10 to 15 mm.

In a further embodiment the overall length of the graft ranges between 10 cm+4×R₀+1 cm and 15 cm+4×R₀+1.5 cm.

The volume of the superior segment 3 is in the range of 53 cm³ to 71 cm³.

The volume of the inferior segment 10 is in the range of 24 cm³ to 61 cm³.

In a special embodiment the medial segment 5 has a tubular structure.

In a further embodiment the biocompatible flexible material has a compliance in the range of 1.8×10⁻² to 2.0×10⁻² mmHg and typically of 1.9×10⁻² mmHg.

In a further embodiment the elastic modulus of the biocompatible flexible material is in the range of 800 to 900 MPa.

The graft according to the invention is suitable for curing the Marfan and Loeys-Dietz syndrome.

According to the invention a method for preserving the native aortic valve is disclosed comprising the following steps:
a) Aortic root as well the ascending aorta are dissected free from the surrounding tissue.
b) At the level of the aortic root it is imperative to separate the aortic root base from the surrounding structure.
c) Following the dissection of the aortic root from the surrounding tissue, the ascending aorta is opened in circular manner about 1.5 cm above the sinotubular junction.
d) As next the excision of three sinuses is performed.
e) Approximately 5 mm of the tissue wall is left, at the level of the intervalvular triangles as well at the nadir of each sinus, superior to the valve attachment in order to provide graft to the aortic root suture.
f) The opening of both coronary vessels is suspended.
g) At this point the aortic root base 23 is separated from surrounding tissue, in order to provide adaptation with the inferior segment 10 of the prosthetic aortic root replacement graft according to one of the claims 2 to 33.
h) Following that, the pledget-mounted sutures are placed, from the inside of the aortic root base, toward the outside of the aortic root base.
i) Between the nadirs of the left coronary sinus and nadirs of the non-coronary sinus the sutures are placed in a straight line, just superior to the left atrium and mitral valve transition.
j) From the middle of the non-coronary to the nadir of the right coronary sinus, the stitches are placed in a semilunar fashion, in the form following the attachment of the leaflets at the non-coronary and the right coronary sinus.
k) From the nadir of the right coronary sinus to the nadir of the left coronary is in a straight line, corresponding to the border of the aortic root base.
l) Following in each commissure, staying sutures are positioned and three commissures, the staying sutures are pulled up, in vertical straight direction. Doing so the intervalvular triangles are positioned in natural vertical direction.
m) This step enables the surgeon to estimate the optimal diameter of the sinotubular junction and with this the size of the prosthetic aortic root replacement graft to be implanted
n) Holding the three commissures in vertical position, the diameter of the sinotubular junction is estimated with a circular graft sizer 17. The radius of the graft sizer 17 is the same as the radius of the inferior segment 10 and the superior segment of the prosthesis. After coronary sinuses were excised, all three intervalvular triangles are pulled in straight vertical direction. Following that the sizer 17, is placed over the three commissures, and the radius at the sinotubular junction is evaluated. The sizer 17 is positioned in horizontal direction just at the level of the three commissures. After the corresponding radius is defined with the sizer 17, a prosthetic aortic root replacement graft according to one of the claims 2-33 with the same diameter as the inferior segment 10 and the superior segment 3 is chosen and implanted.
o) The sizer 17 is just placed above the top of each vertically pulled intervalvular triangle. The radius of the virtual circle has to match 1:1 to the prosthesis sizer 17.
p) The corresponding prosthetic aortic root replacement graft according to one of the claims 2-33 is now chosen.
q) The three commissures and with this the intervalvular triangles are in following step pulled trough the hollow inner space of the graft.
r) The pledged sutures are now traversing the inferior circular segment 10 of the graft, from inside to outside. Finally the sutures are tied down, consequently the inferior part 10 is pulled down to the level of the aortic root. In this position, it serves as stabilization of the aortic root base, and prevents any dilatation in future.
s) At final stage the intervalvular triangles are sutured with running suture to the tongues 7 of the middle segment 5 from its deepest point up to the superior edge 13 of each tongue 7, in semilunar fashion.
t) The three commissures are also sutured with each with one single pledged suture to the superior edge 13 of the tongue 7.
u) Then both coronary artery ostia are re-implanted to the bulges 12 of the medial segment 5.
v) The superior segment 3 with its free edge is sutured to the ascending aorta or to the aortic arch.

### BRIEF DESCRIPTION OF THE DRAWINGS

Several embodiments of the invention will be described in the following by way of example and with reference to the accompanying drawings in which:

FIG. 1 illustrates a schematic perspective view of an embodiment of the device according to the invention;

FIG. 2 shows an exploded view of the embodiment of FIG. 1.

FIG. 3 illustrates the dimensional relationship of the various parts of the embodiment of FIG. 1.

FIG. 4 shows a portion of the medial segment of an alternative embodiment of the invention.

FIG. 5 indicates the location of various cross sections A-A, B-B, C-C, D-D, E-E and F-F through the embodiment of FIG. 1 and represented in detail in section sectional views according to FIGS. 6-11, respectively.

FIG. 6-11 show detailed sectional views at the cross sections A-A, B-B, C-C, D-D, E-E and F-F, respectively.

FIG. 12 shows a sizer to be used in the method for preserving the native aortic valve from profile.

FIG. 13 shows a superior view of the sizer according to FIG. 12, with the dimensions R₀, R₁ and R₂.

FIG. 14 shows the process of measurement of the prosthesis with the aid of the sizer according to FIGS. 12/13.

### DETAILED DESCRIPTION OF THE INVENTION

The following example clarifies the invention further in more detail.

The aortic root prosthesis according to the invention has three segments:
(i) a tubular superior segment 3 which has the shape of a straight cylinder-like part;
(ii) a medial segment 5, with three bulges 12 simulating the natural morphology of three sinuses of Valsalva and—staggerdly arranged between the bulges 12—tongues 7 having an approximate triangular shape and serving for fixation of the natural intervalvular triangles and the commissures; and
(iii) an inferior segment 10 having the shape of a straight cylinder and serving for fixation of the prosthesis at the aortic root base. All three segments 3, 5, 10 of the prosthesis are arranged along a common longitudinal axis 1, and are made of woven polyester graft. The woven structure of the superior segment 3, of the inferior segment 10 and of the three tongues 7 of the medial segment are in horizontal direction that is perpendicular on the axe of the prosthesis. The prosthesis is preferably made of polyester, such as sold under the trademark DACRON and includes a plurality of integrally formed as Z-folds extending around in circumferential fashion around the tube. This with exception of the three sinuses, where the folds in graft are in vertical direction. This horizontal arrangement at tubular segment 3 allows vertical expansion that is expansion along the long axis of the prosthesis. Vertical arrangement of the folds such at the three sinuses of the prosthesis, in contras aloud horizontal expansion. So, tube part, the sinus part and intervalvular triangles are formed from double waved polyester material that is known under trademark DACRON and Hemishield. Whereas in case of sinus the fold in Dacron graft represents vertical direction. To add is that the three triangles like shapes 12 are made of as well of the same polyester material (DACRON) implementing the wire reinforcement 5. Alternatively, the triangular posts 12 may be made of double polyester layer with wire enforcement in between two layers. This allows a vertical expansion of each of these structural elements 3, 10 and 7. Whereas in the woven structure of the three bulges 12 of the medial segment 5 are in vertical direction that is parallel to the axis and as such allows expansion in horizontal direction.

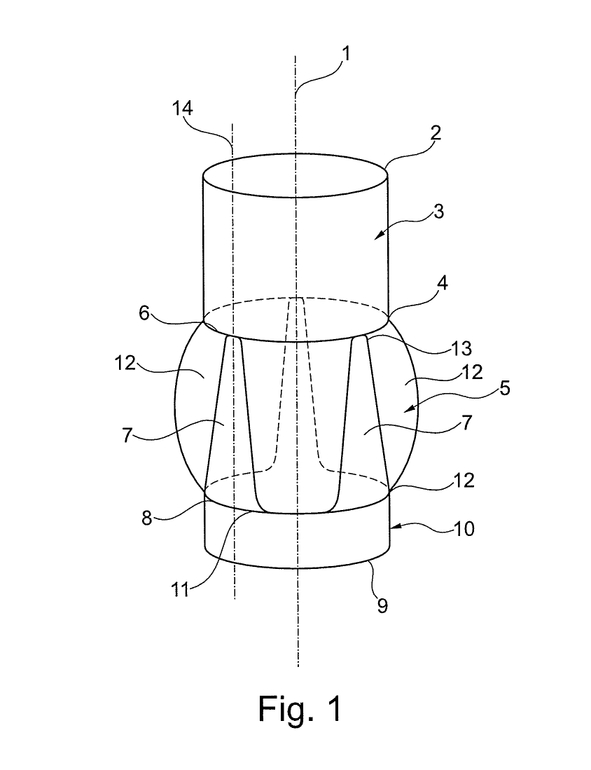

As shown in FIG. 1 the superior segment 3 is a straight tubular/cylinder like structure, with an upper edge 2 serving for suture to the aortic arch. The lower edge 4 and upper edge 2 have a circle like structure and are of the same diameter. The lower edge 4 is mounted (connected to) by the manufacturer to the superior segment, or superior border 3 of the medial segment 5 of the prosthesis 5, 7. With another words, the inferior edge 4 is transient to the superior edges of the triangular posts 5 and to the superior edges of the bulgings 7. This attachment is performed during production process by manufacturer.

The transition between the medial segment 5 of the prosthesis and the straight tubular part of the superior segment 3 is smooth and aligned at the lower edge 4 of the superior segment 3.

The medial segment 5 of the prosthesis has two different kinds of components.

The first kind of components comprises three outwardly flaired bulges 12. These outwardly flaired bulges 12—after the implantation—simulate or mimic the anatomy of the natural sinuses of Valsalva, and bulge outward in order to provide horizontal flexibility after implantation. This function is provided by horizontally directed Z folds of the material. The three bulges 12 have the same dimensions. The lower edge 8 of the three bulges 12 is in smooth transition to inferior segment 10.

The three bulges 12 serve also for re-implantation of the coronary vessels.

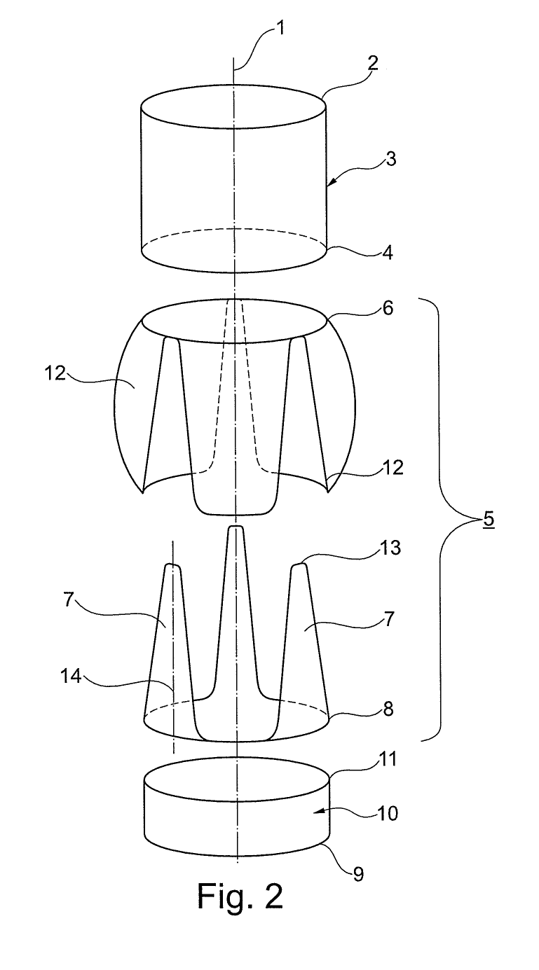

The second kind of components of the medial segment 5 comprises three straight, in vertical direction oriented, triangular like tongues 7. As shown in FIG. 2 the central axis 14 of the tongues 7 are parallel to the middle axis 1 of the prosthesis.

These three tongues 7 are not flaired and do not follow the bulging shape of the three bulges 12. They are rather straight and show vertical expansion similar to the superior segment 3. Therefore the tongues 7 are extending in the same imaginary cylindrical surface as the surface of the superior segment 3.

The transition between the bulges 12 and the tongues 7 is smooth, and aligned.

The lower edge 8 of the three bulges 12 is in smooth transition to the inferior part of the triangles 7.

The tongues 7 serve, after implantation of the prosthesis, as suture platform for the native intervalvular triangles and the three commissures. In this form the natural vertical, almost straight alignment of the natural intervalvular triangles and of three commissures (and with this the attachment of the aortic valve) is assured. The superior edge 13 of the tongues 7 reaches up to the lower edge 4 of the superior segment 3. The three tongues 7 have an internal stiffening system, in order to provide straight alignment during the whole cardiac cycle.

The third part of the prosthesis comprises an inferior segment 10 having the shape of a straight cylinder with its upper edge 11 in smooth transition to the lower edge 8 of the medial segment 5. Its lower edge 9 is circular with the same radius as the upper edge 11. The inferior segment 10 serves for fixation at the aortic root base.

In FIG. 2 all structural elements of the prosthesis are represented as separate elements.

### Superior Segment 3

It has the form of a straight cylinder with an upper edge 2 and lower edge 4. Both edges 2, 4 are parallel. The expansion direction of this straight part is in vertical direction corresponding to the middle axis 1.

### Medial Segment 5

The lower edge 4 of the superior segment 3 is smoothly transient to the upper edge 6 of the medial segment 5 with the three bulges 12 and with superior edges 13 of the three tongues 7.

The bulges 12 have its upper edges 6 which are circular and have the same radius as the lower edge 4 of the superior segment 3. The three bulges 12 are of the same dimensions. One bulge 12 is destined for the left coronary sinus, one for the non-coronary sinus and one for the right coronary sinus. The space between each bulge 12 corresponds to the dimensions of the three tongues 7 and fits the shape of each tongue 7; the transition between the bulges 12 and the tongues 7 is being smooth. The inferior or the deepest part of the bulges 12 corresponds to the deepest point of each natural sinus and is transient to the lower edge 8 of the tongues 7. The three bulges 12 are expandable in horizontal direction (radially with respect to the middle axis 1).

The three tongues 7 of the medial segment 5 of the prosthesis have a triangular shape that fits exactly in between the bulges 12. The three tongues 7 are of the same shape, and correspond to the natural intervalvular triangles. One tongue 7 is destined for the anterior one for the left and one for the right. These tongues 7 serve for suturing the natural intervalvular triangles, in order to provide natural almost vertical position of the leaflets attachment. The superior edge 13 of the tongues 7 is for the fixation/suture of the three commissures. And the body of the tongues 7 is for suturing the intervalvular triangle to the prosthesis.

The lower edge 8 of the medial segment 5 is circular and has the same shape and radius as the upper edge 11 of the inferior segment 10.

The inferior segment 10 of the prosthesis according to the invention has the shape of a straight circular cylinder and is of the same structure as the superior segment 3. It is expandable in vertical direction and serves for suture of the prosthesis at the aortic root base. Its upper edge 11 is continuous with the lower edge 8 the medial segment 5. Its lower edge 9 and upper edge 11 are parallel and, have the same radius as the superior segment 3.

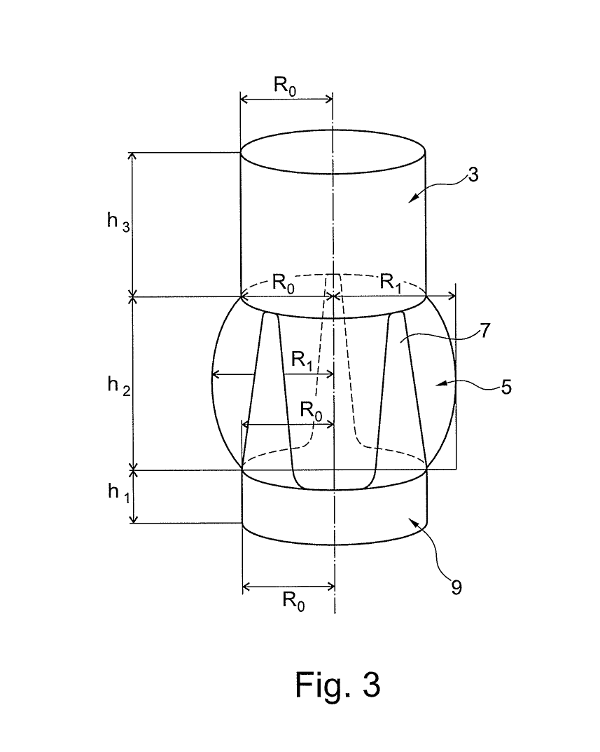

The dimensional relations of the prosthesis according to FIG. 1 are shown in FIG. 3 and can be summarized as follows:
a) R₀ is the radius the hollow cylinder which forms the superior segment 3 and the inferior segment 10;
b) R₁ is the radius of the tubular sinus part (bulges 12) of the prosthesis in the middle of the middle segment 5. This is the distance between the middle axis of the prosthesis and the maximal bulging of the medial segment 5;
c) The relation between R₀ and R₁ is preferably as follows: R₁=R₀+(0.2×R₀);
d) h₁ is the height of the inferior segment 10 and is preferably maximal 1 cm; and
e) h₂ is the height of the medial segment 5, its relation to R₀ is preferably as follows: h₂=4×R₀.

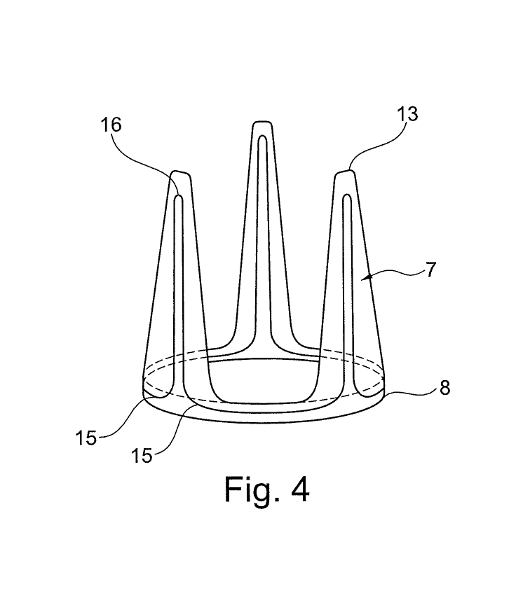

The tongues 7 of the medial segment 5 of a another embodiment of the prosthesis is shown in FIG. 4

The tongues 7 which have a triangular shape are reinforced by wire-like elements 15 being positioned in the interior of the tongues 7. The wire-like elements 15 have a semilunar shape, similar to the basic structure of the tongue 7, which is again the same as the structure of the natural aortic valve attachment. The wire-like elements 15 run from each individual superior edge 7 of the tongue 7 in vertical direction downstream toward the base of the individual tongue 7. Reaching the base of the individual tongue 7 it turns from vertical direction toward the horizontal direction toward the opposite tongue 7.

It then reaches the deepest point of the neighbor tongue 7. Here it curves again toward the superior edge 13. At the top of each tongue 7 the tips of the wire-like elements 15 are shaped in a simple curve. This may also be realized by means of separate U shaped wires 16 connecting two upstreaming wire-like elements 15.

The frame supporting the three tongues 7 may be fabricated by three individual semilunar wire-like elements 15. The cross-section thickness may be different; however it is purposeful to be constant for all three wire-like elements 15

The tissue of the triangular part comprises the reinforcement frame in itself. The wires 16 are incorporated into the tissue. Alternatively the three tongues 7 may be manufacture as double layer tissue structure, but preferably the wires 16 are integrated into the tissue.

However, it is desirable to have enough tissue material for suturing/fixing the native intervalvular triangles with surgical suture

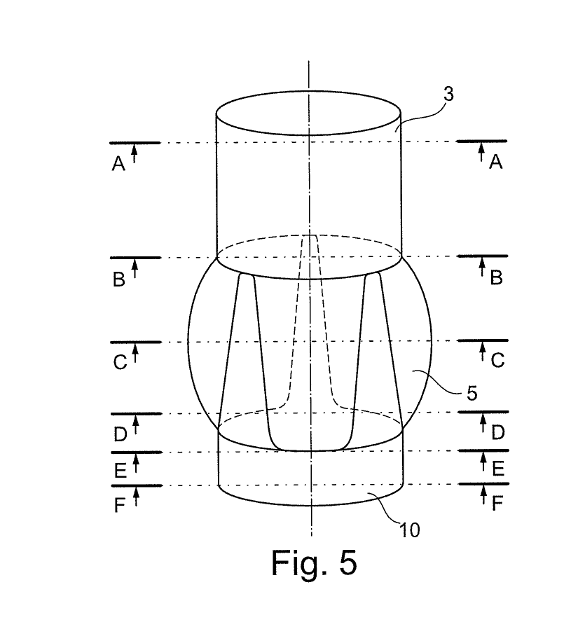

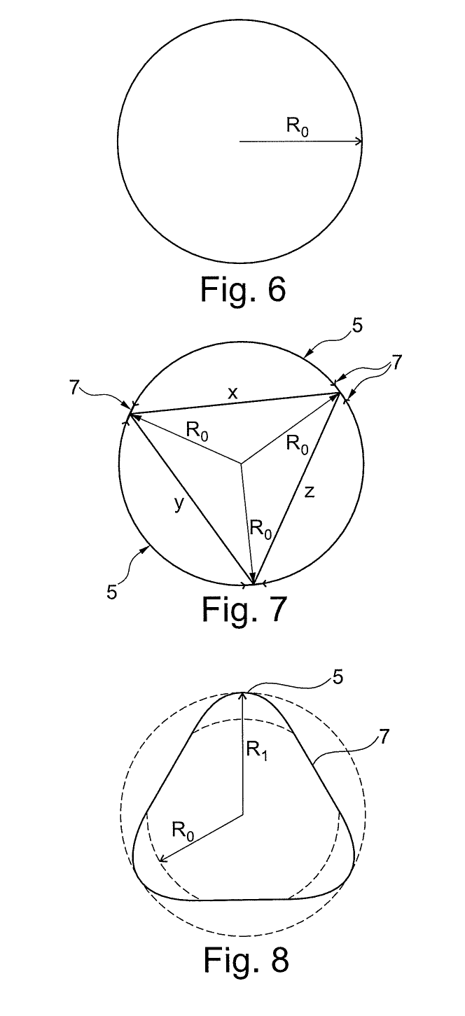

In order to better understand the construction of the prosthesis according to FIG. 1 a number of cross-sections are indicated in FIG. 5 and shown in detail in FIGS. 6-11.

Level A represents a cross section of the superior segment 3, where R₀ is the radius of the straight cylinder representing the superior segment 3 and is shown in FIG. 6.

Level B is at the lower edge 4 of the superior segment 3 which coincides with the upper edge 6 of medial segment 5 as shown in FIG. 7. x, y and z mark the distances between the superior edges 13 of the tongues 7. The distances x, y and z have preferably the same length. The almost upper parts of bulges 12 have here the same radius as the intervalvular tongues 7, namely R₀.

Level C is at the middle of the three bulges 12 (where the prosthesis has is major radial dimension) and is shown in FIG. 8. Here R₀ corresponds to the diameter of the superior segment 3 (being a straight cylinder) and R₁ is the diameter measured from the middle axis 1 of the prosthesis till the maximal convexity of the prosthesis. The bulges 12 at this location have preferably a radius of R₁=R₀+(0.2×R₀).

Level D is at the inferior part of the medial segment 5 and is shown in FIG. 9. Here the bulges 12 and the intervalvular tongues 7 are seen. At this level the circumference of the graft is equally built up from the triangular spots 7 and from bulgings 12. Consequently each individual triangular as well each individual bulging represents ⅙ of the whole circumference.

Level E shown in FIG. 10 is at the upper edge 11 of the inferior segment 10 having the form of a straight cylinder where the radius is R₀.

Level F shown in FIG. 11 is at the lower edge 9 of the inferior segment 10 having the shape of a straight cylinder.

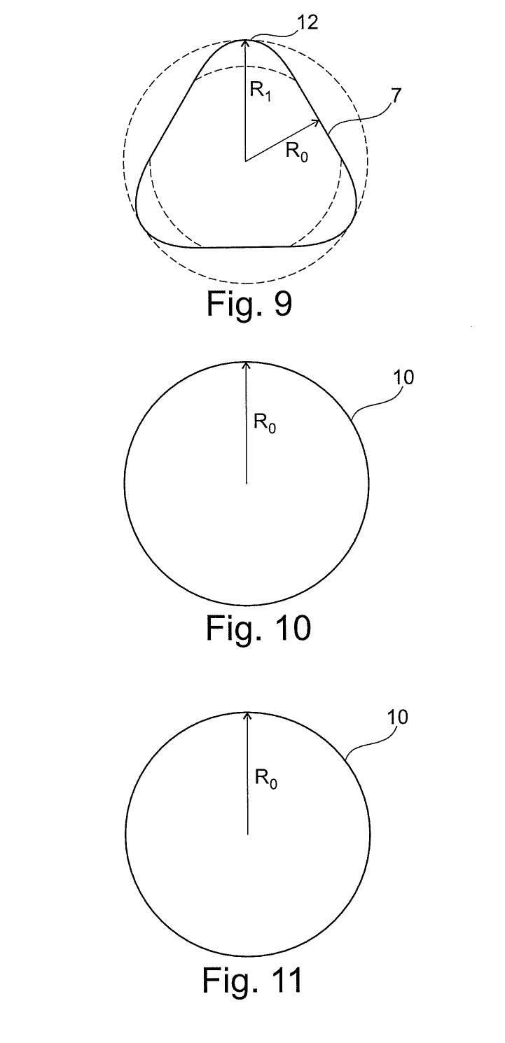

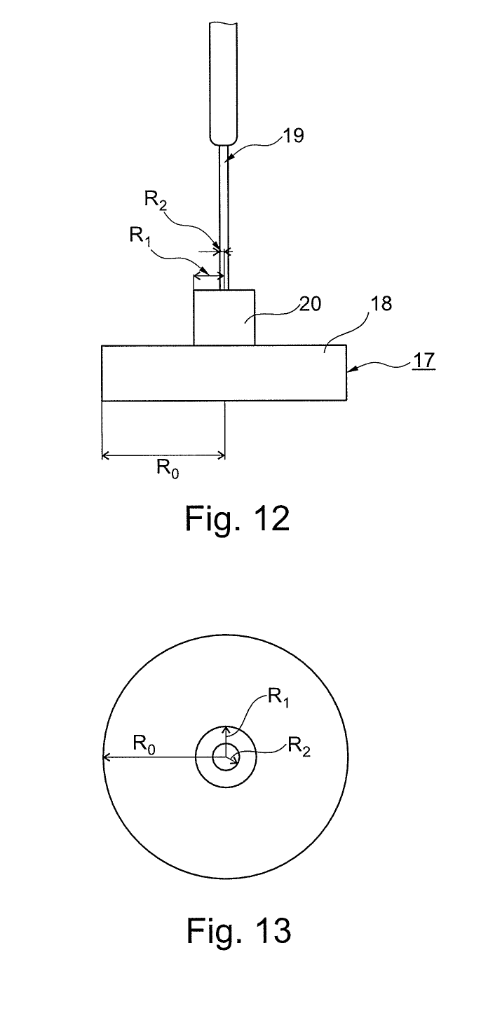

In FIGS. 12 and 13 a sizer 17 for performing the method for preserving the native aortic valve is represented. The cylinder structure 18 is not a hollow part. Its radius corresponds to the radius of the superior segment 3 and the inferior segment 10 of the prosthesis according to the invention. It is a cylinder-like structure with a height fixed at 2 cm. The hand holder 19 is fixed to the cylinder structure 17 with an intermediate segment 20.

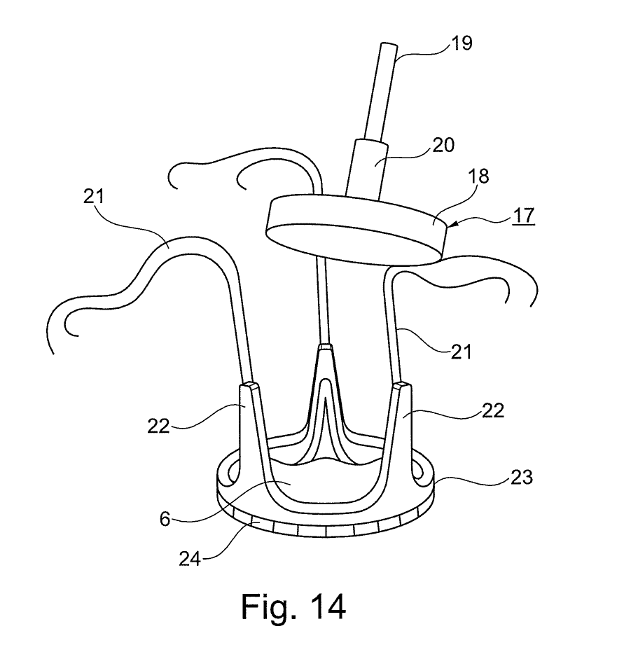

FIG. 14 is showing the process of measurement with the sizer 17. After Valsava sinuses were excised, all three intervalvular triangles 22 are pulled in straight vertical direction, by using the stay sutures 21, positioned just above of the three commissures. Following that, the sizer 17 is placed over the three commissures, and the radius at the sinotubular junction is evaluated. The sizer 17 is positioned in horizontal direction just at the level of the three commissures. After the corresponding radius has been defined with the sizer 17, a prosthetic aortic root replacement graft according to the invention with the same diameters for the inferior segment 10 and the superior segment 3 is chosen and implanted. The position of the staying sutures 21 is to be noted as marked in FIG. 14.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

What is claimed is:

1. A prosthetic aortic root replacement graft for preserving a native aortic valve, said prosthetic aortic root replacement graft comprising a biocompatible flexible material including
a) a tubular superior segment having an upper free edge for anastomosis of the prosthesis to an aortic arch, and a lower edge;
b) a hollow medial segment having an upper edge that is connected to the lower edge of the superior segment, and a lower edge; and
c) optionally a tubular inferior segment;
wherein the medial segment comprises three tongues tapering towards the upper edge and three bulges, wherein the bulges and the tongues are in an alternating arrangement.

2. The aortic root prosthesis graft according to claim 1, wherein the tubular inferior segment is present, and wherein the tubular inferior segment is provided with a free lower edge for fixation at an aortic root base upper surface, and an upper edge that is connected to the lower edge of the medial segment.

3. The aortic root prosthesis graft according to claim 2, wherein a middle axis of the superior, medial and inferior segments define a longitudinal axis of the prosthesis.

4. The aortic root prosthesis graft according to claim 1, wherein the three tongues extend from the lower edge of the medial segment towards the upper edge of the medial segment in a vertical direction.

5. The aortic root prosthesis graft according to claim 1, wherein a middle axis of the superior and medial segments define a longitudinal axis of the prosthesis, and wherein the three tongues run essentially parallel to the longitudinal axis.

6. The aortic root prosthesis graft according to claim 1, wherein the three tongues reach the upper edge of the medial segment.

7. The aortic root prosthesis graft according to claim 1, wherein the tongues are reinforced by wire-like elements, which are positioned in an interior of the tongues.

8. The aortic root prosthesis graft according to claim 7, wherein the wire-like elements have a semilunar shape.

9. The aortic root prosthesis graft according to claim 1, wherein the three tongues are stiffer as compared to the three bulges.

10. The aortic root prosthesis graft according to claim 1, wherein the three tongues are configured for fixation to intervalvular triangles of the native aortic root.

11. The aortic root prosthesis graft according to claim 1, wherein a middle axis of the superior and medial segments define a longitudinal axis of the prosthesis, and wherein the superior and inferior segments are elastically expandable in a direction of the longitudinal axis.

12. The aortic root prosthesis graft according to claim 1, wherein a middle axis of the superior and inferior segments define a longitudinal axis of the prosthesis, and wherein the three bulges of the medial segment are elastically expandable transversally to the longitudinal axis.

13. The aortic root prosthesis graft according to claim 1, wherein the biocompatible flexible material has a textile structure.

14. The aortic root prosthesis graft according to claim 13, wherein the textile structure comprises a polyester material.

15. The aortic root prosthesis graft according to claim 13, wherein the textile structure is impregnated with an absorbable protein.

16. The aortic root prosthesis graft according to claim 1, wherein the biocompatible flexible material comprises a GORETEX material.

17. The aortic root prosthesis graft according to claim 1, wherein the graft exhibits three symmetric planes having a middle axis in common.

18. The aortic root prosthesis graft according to claim 1, wherein the medial segment has a tubular structure.

19. A method for curing a patient afflicted with Marfan and Loeys-Dietz syndrome comprising, connecting an aortic root prosthesis graft according to claim 1 to an aortic arch of the patient.

20. A method for preserving a native aortic valve comprising:
a) dissecting an aortic root and ascending aorta free from surrounding tissue;
b) separating the an aortic root base from the surrounding tissue at a level of the aortic root;
c) following the dissection of the aortic root from the surrounding tissue, opening the ascending aorta in circular manner about 1.5 cm above a sinotubular junction;
d) performing an excision of three sinuses;
e) leaving approximately 5 mm of tissue wall at a level of intervalvular triangles and at a nadire of each sinus, superior to a point of valve attachment;
f) suspending an opening of both coronary vessels;
g) separating the aortic root base from surrounding tissue in order to provide adaptation for an inferior segment of a prosthetic aortic root replacement graft according to claim 2;
h) placing pledget-mounted sutures from an inside of the aortic root base toward the outside of the aortic root base;
i) placing sutures between nadirs of the left coronary sinus and nadirs of the non-coronary sinus in a straight line, just superior to a left atrium and mitral valve transition;
j) placing stitches from a middle of the non-coronary sinus to the nadir of the right coronary sinus, the stitches being placed in a semilunar fashion in a form following attachment of leaflets at the non-coronary and the right coronary sinus;
k) placing stitches from the nadir of the right coronary sinus to the nadir of the left coronary is in a straight line, corresponding to a border of the aortic root base;
l) positioning staying sutures in each of three commissures, and pulling up the staying sutures in a vertical straight direction such that the intervalvular triangles are positioned in a natural vertical direction;
m) while holding the three commissures in the vertical position, estimating the diameter of the sinotubular junction using a circular graft sizer;
n) pulling the three commissures and the intervalvular triangles through the hollow inner space of the graft;
o) tying down the sutures such that the inferior part is pulled down to the level of the aortic root;
p) Asuturing the intervalvular triangles with a running suture to the tongues of the middle segment from a deepest point of the middle segment up to the superior edge of each tongue in semilunar fashion;
q) suturing each of the three commissures with one single pledged suture to the superior edge of the tongue;
r) re-implanting both coronary artery ostia to the bulges of the medial segment; and
s) suturing the superior segment at its free edge to the ascending aorta or to the aortic arch.
